(19) United States
(12) Patent Application Publication
(10) Pub. No.: US 2020/0289520 A1
(43) Pub. Date: Sep. 17, 2020

(54) COMPOSITION AND METHOD FOR TREATING PERIPHERAL T-CELL LYMPHOMA AND CUTANEOUS T-CELL LYMPHOMA

(71) Applicant: Rhizen Pharmaceuticals SA, La Chaux-de-Fonds (CH)

(72) Inventor: Swaroop Kumar Venkata Satya Vakkalanka, La Chaux-de-Fonds (CH)

(21) Appl. No.: 16/762,870

(22) PCT Filed: Dec. 5, 2018
(86) PCT No.: PCT/IB2018/059680

§ 371 Date: May 8, 2020

(30) Foreign Application Priority Data
Dec. 6, 2017 (IN) 201741043740

(51) Int. Cl. A61K 31/52; A61K 45/06; A61P 35/00; A61K 9/00

(52) U.S. Cl. CPC A61K 31/52; A61K 9/0053; A61P 35/00; A61K 45/06

(57) ABSTRACT

The present invention relates to the use of a dual selective PI3K delta and gamma protein kinase inhibitor, such as (S)-2-(1-((9H-purin-6-yl)amino)propyl)-3-(3-fluorophenyl)-4H-chromen-4-one (Compound (A), also known as tenalisib) or a pharmaceutically acceptable salt thereof or a pharmaceutical composition containing such an inhibitor for the treatment of peripheral T-cell lymphoma (PTCL) and cutaneous T-cell lymphoma (CTCL). 34 139095.00100/115268675v.1

The present invention claims the benefit of Indian Provisional Application No. 201741043740, filed 6 Dec. 2017, which is hereby incorporated by reference in its entirety.

### FIELD OF THE INVENTION

The present invention relates to the use of a dual selective PI3K delta and gamma protein kinase inhibitor, such as (S)-2-(1-((9H-purin-6-yl)amino)propyl)-3-(3-fluorophenyl)-4H-chromen-4-one (Compound (A), also known as tenalisib) or a pharmaceutically acceptable salt thereof or a pharmaceutical composition containing such an inhibitor for the treatment of peripheral T-cell lymphoma (PTCL) and cutaneous T-cell lymphoma (CTCL).

### BACKGROUND OF THE INVENTION

Lymphoma is the most common blood cancer. The two main forms of lymphoma are Hodgkin lymphoma and non-Hodgkin lymphoma (NHL). Lymphoma occurs when cells of the immune system called lymphocytes, a type of white blood cell, grow and multiply uncontrollably. Cancerous lymphocytes can travel to many parts of the body, including the lymph nodes, spleen, bone marrow, blood, or other organs, and form a mass called a tumor. The body has two main types of lymphocytes that can develop into lymphomas: B-lymphocytes (B-cells) and T-lymphocytes (T-cells). T-cell lymphomas account for approximately 15 percent of all NHLs in the United States. There are many different forms of T-cell lymphomas, some of which are extremely rare. Most T-cell lymphomas can be classified into two broad categories: aggressive (fast-growing) or indolent (slow-growing).

Peripheral T-cell lymphoma (PTCL) consists of a group of rare and usually aggressive (fast-growing) NHLs that develop from mature T-cells. Most T-cell lymphomas are PTCLs, which collectively account for about 10 percent to 15 percent of all NHL cases in the United States.

PTCLs are sub-classified into various subtypes, each of which are typically considered to be separate diseases based on their distinct clinical differences. Most of these subtypes are very rare; the three most common subtypes of PTCL, peripheral T-cell lymphoma not otherwise specified (PTCL-NOS), anaplastic large-cell lymphoma (ALCL), and angioimmunoblastic T-cell lymphoma (AITL), account for approximately 70 percent of all PTCLs in the United States.

Peripheral T-cell lymphoma not otherwise specified (PTCL NOS) refers to a group of diseases that do not fit into any of the other subtypes of PTCL. PTCL-NOS is the most common PTCL subtype, making up about one quarter of all PTCLs. It is also the most common of all the T-cell lymphomas. The term PTCL can be confusing as it can refer to the entire spectrum of mature T-cell lymphomas, but it can also refer to the specific PTCL-NOS subtype. Although most patients with PTCL-NOS are diagnosed with their disease confined to the lymph nodes, sites outside the lymph nodes, such as the liver, bone marrow, gastrointestinal tract, and skin, may also be involved. This group of PTCLs is aggressive and requires combination chemotherapy upon diagnosis.

Anaplastic large-cell lymphoma (ALCL) is an aggressive T-cell lymphoma, accounting for about three percent of all lymphomas in adults (about 15 percent to 20 percent of all PTCLs) and between 10 percent and 30 percent of all lymphomas in children. ALCL can appear in the skin or in other organs throughout the body (systemic ALCL). ALCL has several different subtypes, each with different expected outcomes and treatment options.

Angioimmunoblastic T-cell lymphoma (AITL) is an aggressive T-cell lymphoma that accounts for about two percent of all NHL cases (about 10 percent to 15 percent of all PTCLs) in the United States. This type of lymphoma often responds to milder therapies, such as steroids, although it often progresses and requires chemotherapy and other medications. In advanced cases, bone marrow transplantation may be used.

Cutaneous T-cell lymphomas (CTCL) are a group of lymphomas that originate in the skin. CTCLs are a subset of PTCL because they are lymphomas of mature T-cells. However, these lymphomas are generally less aggressive, have a different prognosis, and have different treatment approaches than the aggressive PTCLs.

Enteropathy-type T-cell lymphoma is an extremely rare subtype of PTCL that appears in the intestines and is strongly associated with celiac disease.

Nasal NK/T-Cell lymphoma involves natural killer (NK) cells, which are closely related to and often have features that overlap with T-cells. Although this aggressive lymphoma is very rare in the United States, it is more common in Asia and parts of Latin America, leading researchers to suspect that some ethnic groups may be more prone to this cancer. This type of lymphoma is associated with the Epstein-Barr virus and often involves the nasal area, trachea, gastrointestinal tract, or skin.

Hepatosplenic gamma-delta T-cell lymphoma is an extremely rare and aggressive disease that starts in the liver or spleen.

Many new drugs are being studied in clinical trials for the treatment of PTCL, including alemtuzumab (Campath), alisertib (MLN8237), bortezomib (Velcade), brentuximab vedotin (Adcetris), carfilzomib (Kyprolis), dasatinib (Sprycel), E7777, fludarabine (Fludara), lenalidomide (Revlimid), nelfinavir (Viracept), panobinostat (LBH-589), pralatrexate (Folotyn), romidepsin (Istodax), temsirolimus (Torisel) and vorinostat (Zolinza). Vaccine therapy is also being investigated in clinical trials.

One of the most common forms of T-cell lymphoma is cutaneous T-cell lymphoma (CTCL), a general term for T-cell lymphomas that involve the skin. CTCL also can involve the blood, the lymph nodes, and other internal organs. Symptoms can include dry skin, itching (which can be severe), a red rash, and enlarged lymph nodes. The disease affects men more often than women and usually occurs in men in their 50s and 60s. Most patients with CTCL experience only skin symptoms, without serious complications; however, approximately 10 percent of those who progress to later stages develop serious complications. Early stage CTCL is typically indolent; some patients with early-stage CTCL might not progress to later stages at all, while others might progress rapidly, with the cancer spreading to lymph nodes and/or internal organs.

CTCL describes many different disorders with various symptoms, outcomes, and treatment considerations. The two most common types are mycosis fungoides and Sézary syndrome.

Mycosis fungoides is the most common type of CTCL, with approximately 16,000 to 20,000 cases across the United States, accounting for half of all CTCLs. The disease looks different in each patient, with skin symptoms that can appear as patches, plaques, or tumors. Patches are usually flat, possibly scaly, and look like a rash; plaques are thicker, raised, usually itchy lesions that are often mistaken for eczema, psoriasis, or dermatitis; and tumors are raised bumps, which may or may not ulcerate. It is possible to have more than one type of lesion. A medical history, physical exam, and skin biopsy are used for diagnosis. A physician will examine lymph nodes, order various blood tests, and may conduct other screening tests, such as a chest x-ray or a computed axial tomography (CAT) scan. Scans are usually not needed for those with the earliest stages of the disease. Mycosis fungoides is difficult to diagnose in its early stages because the symptoms and skin biopsy findings are similar to those of other skin conditions.

Sezary syndrome is an advanced, variant form of mycosis fungoides, which is characterized by the presence of lymphoma cells in the blood. Extensive thin, red, itchy rashes usually cover over 80 percent of the body. In certain patients, patches and tumors appear. Patients may also experience changes in the nails, hair, or eyelids, or have enlarged lymphnodes. Many of the same procedures used to diagnose and stage other types of cutaneous T-cell lymphomas are used in Sézary syndrome. In addition, a series of imaging tests may be needed to determine if the cancer has spread to the lymph nodes or other organs (although that uncommonly occurs). These tests may include a CAT scan, a positron emission tomography (PET) scan, and/or a magnetic resonance imaging (MRI) scan. A bone marrow biopsy may also be done, but is usually not necessary.

Many treatments at various stages of drug development are currently being tested in clinical trials and for various stages of CTCL, including everolimus (Afinitor), lenalidomide (Revlimid), brentuximab vedotin (Adcetris), panobinostat, forodesine, APO866, and KW0761.

Phosphoinositide-3 kinase (PI3K) belongs to a class of intracellular lipid kinases that phosphorylate the 3 position hydroxyl group of the inositol ring of phosphoinositide lipids (PIs) generating lipid second messengers. While alpha and beta isoforms are ubiquitous in their distribution, expression of delta and gamma is restricted to circulating hematogenous cells and endothelial cells. Unlike PI3K-alpha or beta, mice lacking expression of gamma or delta do not show any adverse phenotype indicating that targeting of these specific isoforms would not result in overt toxicity.

Recently, targeted inhibitors of the phosphoinositide-3-kinase (PI3K) pathway have been suggested as immunomodulatory agents. This interest stems from the fact that the PI3K pathway serves multiple functions in immune cell signaling, primarily through the generation of phosphatidylinositol (3,4,5)-trisphosphate (PIP3), a membrane bound second messenger. PIP3 recruits proteins to the cytoplasmic side of the lipid bilayer, including protein kinases and GTPases, initiating a complex network of downstream signaling cascades important in the regulation of immune cell adhesion, migration, and cell-cell communication.

The four class I PI3K isoforms differ significantly in their tissue distribution. PI3Kα and PI3Kβ are ubiquitous and activated downstream of receptor tyrosine kinases (RTK), whereas PI3K δ and PI3K γ are primarily limited to hematopoietic and endothelial cells, and are activated downstream of RTKs, and G protein coupled receptors (GPCR), respectively. Mouse genetic studies have revealed that PI3Kα and PI3Kβ are essential for normal development, whereas loss of PI3K δ and/or PI3K γ yields viable offspring with selective immune deficits.

Reviews and studies regarding PI3K and related protein kinase pathways have been given by Liu et. al., Nature Reviews Drug Discovery, 8, 627-644, 2009); Nathan T. et. al., Mol Cancer Ther., 8(1), 2009; Marone et, al., Biochimica et Biophysica Acta, 1784, 159-185, 2008 and Markman et. al., Annals of Oncology Advance Access, published August 2009. Similarly reviews and studies regarding role of PI3K δ and γ have been given by William et. al., Chemistry & Biology, 17, 123-134, 2010 and Timothy et. al. J. Med. Chem., 55 (20), 8559-8581, 2012. All of these literature disclosures are hereby incorporated by reference in their entirety.

Despite some progress made in the area of treatment in peripheral T-cell lymphoma (PTCL) and cutaneous T-cell lymphoma (CTCL), challenges remain in the treatment, side effects and desired clinical benefits of them. Accordingly, there still remains an unmet need for drugs for the treatment of PTCL and CTCL.

### SUMMARY OF THE INVENTION

In one aspect, the present invention relates to the use of a dual selective PI3K delta and gamma inhibitor for treating peripheral T-cell lymphoma (PTCL) and cutaneous T-cell lymphoma (CTCL).

The inventors surprisingly found that the dual selective PI3K delta and gamma inhibitor (S)-2-(1-((9H-purin-6-yl)amino)propyl)-3-(3-fluorophenyl)-4H-chromen-4-one (Compound (A) or tenalisib, shown below) or a pharmaceutically acceptable salt thereof exhibits excellent activity against PTCL and CTCL.

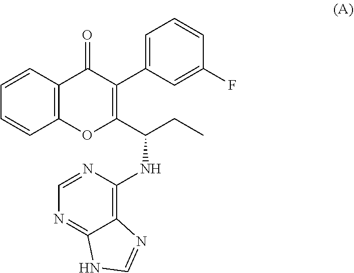 [SMILES: CC[C@H](Nc1ncnc2[nH]cnc12)c1oc2ccccc2c(=O)c1-c1cccc(F)c1]

One embodiment is the use of a dual selective PI3K delta and gamma inhibitor for the treatment of peripheral T-cell lymphoma (PTCL) or cutaneous T-cell lymphoma (CTCL). A preferred embodiment is the use of (S)-2-(1-((9H-purin-6-yl)amino)propyl)-3-(3-fluorophenyl)-4H-chromen-4-one or a pharmaceutically acceptable salt thereof for the treatment of peripheral T-cell lymphoma (PTCL) or cutaneous T-cell lymphoma (CTCL).

The dual selective PI3K delta and gamma inhibitor may be administered as a front-line therapy or as a relapsed-refractory therapy for the treatment of a peripheral T-cell lymphoma (PTCL).

The dual selective PI3K delta and gamma inhibitor may be administered as a front-line therapy or as a relapsed-refractory therapy for the treatment of a cutaneous T-cell lymphoma (CTCL).

Another embodiment is a method of treating a peripheral T-cell lymphoma (PTCL) or cutaneous T-cell lymphoma (CTCL) in a subject (preferably a human subject) comprising administering to the subject an effective amount of a dual selective PI3K delta and gamma inhibitor.

A preferred embodiment is a method of treating a peripheral T-cell lymphoma (PTCL) or cutaneous T-cell lymphoma (CTCL) in a subject (preferably a human subject) comprising administering to the subject (preferably a human subject) an effective amount of Compound (A) or a pharmaceutically acceptable salt thereof.

Yet another embodiment is a method of inhibiting PI3K delta and gamma activity in a subject (preferably a human subject) suffering from a Peripheral T-cell lymphoma (PTCL) or Cutaneous T-cell lymphoma (CTCL) by administering to the subject an effective amount of a dual selective PI3K delta and gamma inhibitor. In a preferred embodiment, the dual selective PI3K delta and gamma inhibitor is Compound (A) or a pharmaceutically acceptable salt thereof.

An object of the present invention relates to the uses described herein for the treatment of a subject, in particular of a human subject.

An object of the present invention is the use of Compound (A) or a pharmaceutically acceptable salt thereof for the preparation of a medicament intended for the treatment of a peripheral T-cell lymphoma (PTCL) or cutaneous T-cell lymphoma (CTCL).

Another object of the present invention is the use of Compound (A) or a pharmaceutically acceptable salt thereof for the preparation of a medicament intended for the treatment of a peripheral T-cell lymphoma (PTCL) or cutaneous T-cell lymphoma (CTCL), where the medicament is administered orally.

The dual selective PI3K delta and gamma inhibitor, such as Compound (A) or a pharmaceutically acceptable salt thereof, can be administered to the subject by the oral route, the intravenous route, the intramuscular route, or the intraperitoneal route. In one preferred embodiment, the dual selective PI3K delta and gamma inhibitor is administered orally.

In one embodiment, the dual selective PI3K delta and gamma inhibitor, such as Compound (A) or a pharmaceutically acceptable salt thereof, is administered as a front-line therapy for a peripheral T-cell lymphoma (PTCL).

In another embodiment, the dual selective PI3K delta and gamma inhibitor, such as Compound (A) or a pharmaceutically acceptable salt thereof, is administered as a relapsed-refractory therapy for a peripheral T-cell lymphoma (PTCL).

In one embodiment, the dual selective PI3K delta and gamma inhibitor, such as Compound (A) or a pharmaceutically acceptable salt thereof, is administered as a front-line therapy for a cutaneous T-cell lymphoma (CTCL).

In another embodiment, the dual selective PI3K delta and gamma inhibitor, such as Compound (A) or a pharmaceutically acceptable salt thereof, is administered as a relapsed-refractory therapy for a cutaneous T-cell lymphoma (CTCL).

In yet another embodiment, in any of the uses of the dual selective PI3K delta and gamma inhibitor and methods described herein, the dual selective PI3K delta and gamma inhibitor is used in combination (administered together or sequentially) with an anti-cancer treatment, one or more cytostatic, cytotoxic or anticancer agents, targeted therapy, or any combination or any of the foregoing.

Suitable anti-cancer treatments include, e.g., radiation therapy. Suitable cytostatic, cytotoxic and anticancer agents include, but are not limited to, DNA interactive agents, such as cisplatin or doxorubicin; topoisomerase II inhibitors, such as etoposide; topoisomerase I inhibitors such as CPT-11 or topotecan; tubulin interacting agents, such as paclitaxel, docetaxel or the epothilones (for example, ixabepilone), either naturally occurring or synthetic; hormonal agents, such as tamoxifen; thymidilate synthase inhibitors, such as 5-fluorouracil; and anti-metabolites, such as methotrexate, other tyrosine kinase inhibitors such as gefitinib (marketed as Iressa®) and erlotinib (also known as OSI-774); angiogenesis inhibitors; EGF inhibitors; VEGF inhibitors; CDK inhibitors; SRC inhibitors; c-Kit inhibitors; Her1/2 inhibitors and monoclonal antibodies directed against growth factor receptors such as erbitux (EGF) and herceptin (Her2), and other protein kinase modulators.

Yet another embodiment is Compound (A) or a pharmaceutically acceptable salt thereof for use in the front-line therapy of a peripheral T-cell lymphoma (PTCL).

Yet another embodiment is Compound (A) or a pharmaceutically acceptable salt thereof for use in the relapsed-refractory therapy of a peripheral T-cell lymphoma (PTCL).

Yet another embodiment is Compound (A) or a pharmaceutically acceptable salt thereof for use in the front-line therapy of a cutaneous T-cell lymphoma (CTCL).

Yet another embodiment is Compound (A) or a pharmaceutically acceptable salt thereof for use in the relapsed-refractory therapy of a cutaneous T-cell lymphoma (CTCL).

Yet another embodiment is a pharmaceutical composition for treating a peripheral T-cell lymphoma (PTCL) or cutaneous T-cell lymphoma (CTCL) comprising a dual selective PI3K delta and gamma inhibitor (preferably Compound (A) or a pharmaceutically acceptable salt thereof), and optionally one or more pharmaceutically acceptable carriers or excipients.

In one embodiment, the pharmaceutical composition further comprises one or more cytostatic, cytotoxic or anticancer agents.

In one embodiment, the pharmaceutical composition is useful in combination with one or more anti-cancer treatments, one or more cytostatic, cytotoxic or anticancer agents, targeted therapy, or any combination or any of the foregoing. The dual selective PI3K delta and gamma inhibitor may be used together or sequentially with one or more anti-cancer treatments one or more cytostatic, cytotoxic or anticancer agents, targeted therapy, or any combination or any of the foregoing.

In one preferred embodiment, the pharmaceutical composition of the dual selective PI3K delta and gamma inhibitor (preferably Compound (A) is suitable for oral administration.

In another embodiment, Compound (A) or a pharmaceutically acceptable salt thereof is administered at a dose of about 25 to about 2000 mg, such as a dose of about 25 to about 1600 mg, about 25 to about 1200 mg, about 25 to about 800 mg, about 25 to about 600 mg, or about 25 to about 400 mg.

In yet another embodiment, Compound (A) or a pharmaceutically acceptable salt thereof is administered at a dose of about 50 to about 2000 mg, such as a dose of about 50 to about 1600 mg, about 50 to about 1200 mg, about 50 to about 800 mg, about 50 to about 600 mg, or about 50 to about 400 mg.

In another embodiment, Compound (A) or a pharmaceutically acceptable salt thereof is administered at a dose of about 200 to about 2000 mg, such as a dose of about 200 to about 1600 mg, about 200 to about 1200 mg, about 200 to about 800 mg, about 200 to about 600 mg, or about 200 to about 400 mg.

In another embodiment, Compound (A) or a pharmaceutically acceptable salt thereof is administered at a dose of about 400 to about 2000 mg, such as a dose of about 400 to about 1600 mg, about 400 to about 1200 mg, about 400 to about 800 mg, or about 400 to about 600 mg.

In another embodiment, Compound (A) or a pharmaceutically acceptable salt thereof is administered at a dose of about 25 to about 2000 mg per day, such as a dose of about 50 to about 1200 mg per day or a dose of about 400 to about 800 mg per day or a dose of about 200 to about 400 mg per day. In one embodiment, these daily doses are for oral administration of Compound (A) or a pharmaceutically acceptable salt thereof.

Compound (A) or a pharmaceutically acceptable salt thereof may be administered as a single dose or in divided doses.

In another embodiment, Compound (A) or a pharmaceutically acceptable salt thereof, is administered once daily. In yet another embodiment, Compound (A) or a pharmaceutically acceptable salt thereof is administered twice daily.

In the uses and methods described herein, the subject can be a human subject suffering from relapsed peripheral T-cell lymphoma (PTCL), refractory peripheral T-cell lymphoma (PTCL), or relapsed-refractory peripheral T-cell lymphoma (PTCL).

In the uses and methods described herein, the subject can be a human subject suffering from relapsed cutaneous T-cell lymphoma (CTCL), refractory cutaneous T-cell lymphoma (CTCL), or relapsed-refractory cutaneous T-cell lymphoma (CTCL).

### BRIEF DESCRIPTION OF THE FIGURES

FIG. 1 is a graph of the percent viability of certain T-lymphoma cell lines (namely, Jurkat, MOLT-4, CCRF-CEM, HuT-78, and HuT-102 cells) at various concentrations of Compound (A) as measured by the procedure described in Example 2.

FIG. 2 is a graph of the inhibition of Phospho-AKT (pAKT) in T-cell lymphoma cell lines when in the presence of various concentrations of Compound (A) as measured by the procedure described in Example 2.

FIG. 3 is a graph showing the percent induction of caspase-3 activity in T-lymphoma cell lines (namely, Jurkat, MOLT-4, CCRF-CEM, HuT-78, and HuT-102) at various concentrations of Compound (A) as measured by the procedure described in Example 2.

FIG. 4 is graph showing the percent inhibition of Phospho-AKT (pAKT) in purified malignant T-cells at various concentrations of Compound (A) and LY294002 as measured by the procedure described in Example 3.

FIG. 5 is a bar graph showing the percentage of apoptosis estimated by Annexin V/PI staining in purified malignant T-cells, either untreated or treated with camptothecin or Compound (A) at various concentrations, as measured by the procedure described in Example 3.

FIG. 6 is a graph of tumor volume (mm³) over time in the MOLT-4 Human Leukemia Xenograft Model treated with a vehicle, Compound (A) (50 mg/kg/PO/BID) or Ara-C (50 mg/kg), as measured by the procedure described in Example 4.

FIG. 7a is a bar graph showing the response by individual PTCL patients administered with Compound (A) over a dose range of 200 to 800 mg BID according to the procedure described in Example 5. The indicated dosage amounts were administered twice a day (BID).

FIG. 7b is a bar graph showing the response by individual CTCL patients administered with Compound (A) over a dose range of 200 to 800 mg BID according to the procedure described in Example 5. The indicated dosage amounts were administered twice a day (BID).

FIG. 8 is a waterfall plot graph showing the percentage change in nodal size in PTCL and CTCL patients administered with Compound (A) according to the procedure in Example 5.

### DETAIL DESCRIPTION OF THE INVENTION

Unless defined otherwise, all technical and scientific terms used herein have the same meaning as is commonly understood in the field to which the subject matter belongs. In the event that there is a plurality of definitions for terms herein, those in this section prevail. Where reference is made to a URL or other such identifier or address, it is understood that such identifiers generally change and particular information on the internet comes and goes, but equivalent information is found by searching the internet. Reference thereto evidences the availability and public dissemination of such information.

It is to be understood that the foregoing general description and the following detailed description are exemplary and explanatory only and are not restrictive of any subject matter. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Definition of standard chemistry and molecular biology terms are found in reference works, including but not limited to, Carey and Sundberg “ADVANCED ORGANIC CHEMISTRY 4^{th} edition” Vols. A (2000) and B (2001), Plenum Press, New York and “MOLECULAR BIOLOGY OF THE CELL 5^{th} edition” (2007), Garland Science, New York. Unless otherwise indicated, conventional methods of mass spectroscopy, NMR, HPLC, protein chemistry, biochemistry, recombinant DNA techniques and pharmacology are contemplated within the scope of the embodiments disclosed herein.

Unless specific definitions are provided, the nomenclature employed in connection with, and the laboratory procedures and techniques of, analytical chemistry, and medicinal and pharmaceutical chemistry described herein are those generally used. In some embodiments, standard techniques are used for chemical analyses, pharmaceutical preparation, formulation, and delivery, and treatment of patients. In other embodiments, standard techniques are used for recombinant DNA, oligonucleotide synthesis, and tissue culture and transformation (e.g., electroporation, lipofection). In certain embodiments, reactions and purification techniques are performed e.g., using kits of manufacturer's specifications or as described herein. The foregoing techniques and procedures are generally performed of conventional methods and as described in various general and more specific references that are cited and discussed throughout the present specification.

Additionally, the dual selective PI3K delta and gamma inhibitor described herein, including Compound (A) and pharmaceutically acceptable salts thereof, includes the compound which differ only in the presence of one or more isotopically enriched atoms for example replacement of hydrogen with deuterium.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, and swine; domestic animals such as rabbits, dogs, and cats; and laboratory animals including rodents, such as rats, mice and guinea pigs. Examples of non-mammals include, but are not limited to, birds, and fish. In one embodiment of the methods and compositions provided herein, the mammal is a human.

As used herein, the term “treatment” refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

The term “front-line therapy” refers to the first treatment given for a disease. It is often part of a standard set of treatments, such as surgery followed by chemotherapy and radiation. When used by itself, front-line therapy is the one accepted as the best treatment. If it does not cure the disease or it causes severe side effects, other treatment may be added or used instead. It is also called induction therapy, primary therapy, and primary treatment.

The term “relapsed” refers to disease that reappears or grows again after a period of remission.

The term “refractory” is used to describe when the cancer does not respond to treatment (meaning that the cancer cells continue to grow) or when the response to treatment does not last very long.

“Radiation therapy” or “Radiation treatment” means exposing a patient, using routine methods and compositions known to the practitioner, to radiation emitters such as alpha-particle emitting radionuclides (e.g., actinium and thorium radionuclides), low linear energy transfer (LET) radiation emitters (i.e. beta emitters), conversion electron emitters (e.g. strontium-89 and samarium-153-EDTMP), or high-energy radiation, including, without limitation, x-rays, gamma rays, and neutrons.

The term “acceptable” with respect to a formulation, composition or ingredient, as used herein, means having no persistent detrimental effect on the general health of the subject being treated.

By “pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

Pharmaceutically acceptable salts forming part of this invention include salts derived from inorganic bases such as Li, Na, K, Ca, Mg, Fe, Cu, Zn, and Mn; salts of organic bases such as N,N′-diacetylethylenediamine, glucamine, triethylamine, choline, hydroxide, dicyclohexylamine, metformin, benzylamine, trialkylamine, thiamine, and the like; chiral bases like alkylphenylamine, glycinol, and phenyl glycinol, salts of natural amino acids such as glycine, alanine, valine, leucine, isoleucine, norleucine, tyrosine, cystine, cysteine, methionine, proline, hydroxy proline, histidine, omithine, lysine, arginine, and serine; quaternary ammonium salts of the compounds of invention with alkyl halides, and alkyl sulphates such as MeI and (Me)₂SO₄, non-natural amino acids such as D-isomers or substituted amino acids; guanidine, substituted guanidine wherein the substituents are selected from nitro, amino, alkyl, alkenyl, alkynyl, ammonium or substituted ammonium salts and aluminum salts. Salts may include acid addition salts where appropriate which are, sulphates, nitrates, phosphates, perchlorates, borates, hydrohalides, acetates, tartrates, maleates, citrates, fumarates, succinates, palmoates, methanesulphonates, benzoates, salicylates, benzenesulfonates, ascorbates, glycerophosphates, and ketoglutarates.

The term “pharmaceutical composition” refers to a mixture of a compound of the present invention with other chemical components, such as carriers, stabilizers, diluents, dispersing agents, suspending agents, thickening agents, and/or excipients.

The compound and pharmaceutical compositions described herein can be administered by various routes of administration including, but not limited to, intravenous, oral, aerosol, parenteral, ophthalmic, pulmonary and topical administration.

The term “selective inhibition” or “selectively inhibit” as applied to a biologically active agent refers to the agent's ability to selectively reduce the target signaling activity as compared to off-target signaling activity, via direct or indirect interaction with the target.

The terms “effective amount” or “therapeutically effective amount,” as used herein, refer to a sufficient amount of an agent or a compound being administered which will relieve to some extent one or more of the symptoms of the disease or condition being treated. The result is reduction and/or alleviation of the signs, symptoms, or causes of a disease, or any other desired alteration of a biological system. For example, an “effective amount” for therapeutic uses is the amount of a compound of the present invention required to provide a clinically significant decrease in disease symptoms. In some embodiments, an appropriate “effective” amount in any individual case is determined using techniques, such as a dose escalation study.

The term “carrier,” as used herein, refers to relatively nontoxic chemical compounds or agents that facilitate the incorporation of a compound into cells or tissues.

The terms “pharmaceutically acceptable carrier” and “pharmaceutically acceptable excipient” include, but is not limited to, any and all solvents, dispersion media, coatings, antibacterial and antifungal agents, isotonic and absorption delaying agents, one or more suitable diluents, fillers, salts, disintegrants, binders, lubricants, glidants, wetting agents, controlled release matrices, colorants, flavorings, carriers, excipients, buffers, stabilizers, solubilizers, and any combination of any of the foregoing. Except insofar as any conventional media or agent is incompatible with the active ingredient, its use in the therapeutic compositions of the invention is contemplated. Supplementary active ingredients can also be incorporated into the compositions.

As used herein, the term “dual PI3-kinase δ/γ inhibitor” and “dual PI3-kinase δ/γ selective inhibitor” refers to a compound that inhibits the activity of both the PI3-kinase δ and γ isozyme more effectively than other isozymes of the PI3K family. A dual PI3-kinase δ/γ inhibitor is therefore more selective for PI3-kinase δ and γ than conventional PI3K inhibitors such as CAL-130, wortmannin and LY294002, which are nonselective PI3K inhibitors. The relative efficacies of compounds as inhibitors of an enzyme activity (or other biological activity) can be established by determining the concentrations at which each compound inhibits the activity to a predefined extent and then comparing the results. Typically, the preferred determination is the concentration that inhibits 50% of the activity in a biochemical assay, i.e., the 50% inhibitory concentration or “IC₅₀”. IC₅₀ determinations can be accomplished using conventional techniques known in the art. In general, an IC₅₀ can be determined by measuring the activity of a given enzyme in the presence of a range of concentrations of the inhibitor under study. The experimentally obtained values of enzyme activity then are plotted against the inhibitor concentrations used. The concentration of the inhibitor that shows 50% enzyme activity (as compared to the activity in the absence of any inhibitor) is taken as the IC₅₀ value. Analogously, other inhibitory concentrations can be defined through appropriate determinations of activity. For example, in some settings it can be desirable to establish a 90% inhibitory concentration, i.e., IC₉₀.

In one embodiment, the dual PI3-kinase δ/γ selective inhibitor is a compound that exhibits a 50% inhibitory concentration (IC₅₀) with respect to PI3-kinase δ and γ, that is at least 10-fold lower, at least 20-fold lower, or at least 30-fold lower than the IC₅₀ value with respect to any or all of the other class I PI3K family members. In an alternative embodiment, the dual PI3-kinase δ/γ selective inhibitor is a compound that exhibits an IC₅₀ with respect to PI3-kinase δ and γ that is at least 30-fold lower, at least 50-fold lower, at least 100-fold lower, at least 200-fold lower, or at least 500-fold lower than the IC₅₀ with respect to any or all of the other PI3K class I family members. A dual PI3-kinase δ/γ selective inhibitor is typically administered in an amount such that it selectively inhibits both PI3-kinase δ and γ activity, as described above.

In certain embodiments, the compounds of the present invention exhibit PI3-kinase δ and γ inhibition almost equally (˜1:1) or at a maximum ratio of 1:5, i.e., the compound the of the present invention exhibit almost equal IC₅₀ values for both PI3-kinase δ and γ enzyme, or at most a 3 to 8 fold difference between the two.

### Methods of Treatment and Uses

In the methods of treatment and uses described herein, one or more additional active agents can be administered with Compound (A) or a pharmaceutically acceptable salt thereof. For example, Compound (A) or a pharmaceutically acceptable salt thereof may be used in combination (administered together or sequentially) with one or more anti-cancer treatments such as, e.g., chemotherapy, radiation therapy, biological therapy, bone marrow transplantation, stem cell transplant or any other anticancer therapy, or one or more cytostatic, cytotoxic or anticancer agents or targeted therapy, either alone or in combination, such as, but not limited to, DNA interactive agents, such as fludarabine, cisplatin, chlorambucil, bendamustine or doxorubicin; alkylating agents, such as cyclophosphamide; topoisomerase II inhibitors, such as etoposide; topoisomerase I inhibitors such as CPT-11 or topotecan; tubulin interacting agents, such as paclitaxel, docetaxel or the epothilones (for example ixabepilone), either naturally occurring or synthetic; hormonal agents, such as tamoxifen; thymidilate synthase inhibitors, such as 5-fluorouracil; and anti-metabolites, such as methotrexate; other tyrosine kinase inhibitors such as gefitinib (marketed as Iressa®) and erlotinib (also known as OSI-774); angiogenesis inhibitors; EGF inhibitors; VEGF inhibitors; CDK inhibitors; SRC inhibitors; c-Kit inhibitors; Her1/2 inhibitors, checkpoint kinase inhibitors and monoclonal antibodies directed against growth factor receptors such as erbitux (EGF) and herceptin (Her2); CD20 monoclonal antibodies such as rituximab, ublixtumab (TGR-1101), ofatumumab (HuMax; Intracel), ocrelizumab, veltuzumab, GA101 (obinutuzumab), ocaratuzumab (AME-133v, LY2469298, Applied Molecular Evolution, Mentrik Biotech), PRO131921, tositumomab, veltuzumab (hA20, Immunomedics, Inc.), ibritumomab-tiuxetan, BLX-301 (Biolex Therapeutics), Reditux (Dr. Reddy's Laboratories), and PRO70769 (described in WO2004/056312); other B-cell targeting monoclonal antibodies such as belimumab, atacicept or fusion proteins such as blisibimod and BR3-Fc, other monoclonal antibodies such as alemtuzumab and other protein kinase modulators.

The methods of treatment and uses described herein also include use of one or more additional active agents to be administered with Compound (A), or a pharmaceutically acceptable salt, thereof. For example, CHOP (cyclophosphamide, doxorubicin, vincristine, prednisone); R-CHOP (rituximab-CHOP); hyperCV AD (hyperfractionated cyclophosphamide, vincristine, doxorubicin, dexamethasone, methotrexate, cytarabine); R-hyperCV AD (rituximab-hyperCV AD); FCM (fludarabine, cyclophosphamide, mitoxantrone); R-FCM (rituximab, fludarabine, cyclophosphamide, mitoxantrone); bortezomib and rituximab; temsirolimus and rituximab; temsirolimus and bortezomib (Velcade®); Iodine-131 tositumomab (Bexxar®) and CHOP; CVP (cyclophosphamide, vincristine, prednisone); R-CVP (rituximab-CVP); ICE (iphosphamide, carboplatin, etoposide); R-ICE (rituximab-ICE); FCR (fludarabine, cyclophosphamide, rituximab); FR (fludarabine, rituximab); and D.T. PACE (dexamethasone, thalidomide, cisplatin, adriamycin, cyclophosphamide, and etoposide).

The dual selective PI3K delta and gamma inhibitor, including Compound (A) and pharmaceutically acceptable salts thereof, may also be used in combination (administered together or sequentially) with one or more steroidal anti-inflammatory drugs, non-steroidal anti-inflammatory drugs (NSAIDs) or immune selective anti-inflammatory derivatives (ImSAIDs).

In one embodiment, the dual selective PI3K delta and gamma inhibitor, such as Compound (A) or a pharmaceutically acceptable salt thereof, can also be administered in combination with one or more other active principles useful in one of the pathologies mentioned above, for example an anti-emetic, analgesic, anti-inflammatory or anti-cachexia agent.

In another embodiment, the dual selective PI3K delta and gamma inhibitor, such as Compound (A) or a pharmaceutically acceptable salt thereof, can be combined with a radiation treatment.

In another embodiment, the dual selective PI3K delta and gamma inhibitor, such as Compound (A) or a pharmaceutically acceptable salt thereof, can be combined with surgery including either pre, post, or during a period of surgery.

In any of the methods and uses described herein, the compounds and compositions described herein can be administered simultaneously, separately, sequentially and/or spaced in time.

### Dual Selective PI3K Delta and Gamma Inhibitor

The dual selective PI3K delta and gamma inhibitors may be any known in the art, such as those described in International Publication No, PCT/IB2014/061954 filed on Jun. 4, 2014 (WO 2014/195888) (including Compound (A)), which is hereby incorporated by reference in its entirety.

### Pharmaceutical Compositions

The pharmaceutical compositions described herein may comprise a dual selective PI3K delta and gamma inhibitor (preferably Compound (A) or a pharmaceutically acceptable salt thereof) and optionally one or more pharmaceutically acceptable carriers or excipients.

In one embodiment, the pharmaceutical composition includes a therapeutically effective amount of a dual selective PI3K delta and gamma inhibitor, such as Compound (A) or a pharmaceutically acceptable salt thereof. The pharmaceutical composition may include one or more additional active ingredients, as described herein.

Suitable pharmaceutical carriers and/or excipients may be selected from diluents, fillers, salts, disintegrants, binders, lubricants, glidants, wetting agents, controlled release matrices, colorants, flavorings, buffers, stabilizers, solubilizers, and any combination of any of the foregoing.

The pharmaceutical compositions described herein can be administered alone or in combination with one or more other active agents. Where desired, the dual selective PI3K delta and gamma inhibitor(s) and other agent(s) may be mixed into a preparation or both components may be formulated into separate preparations to use them in combination separately or at the same time.

The pharmaceutical compositions described herein can be administered together or in a sequential manner with one or more other active agents. Where desired, the dual selective PI3K delta and gamma inhibitor and other agent(s) may be co-administered or both components may be administered in a sequence to use them as a combination.

The dual selective PI3K delta and gamma inhibitor and pharmaceutical compositions described herein can be administered by any route that enables delivery of the dual selective PI3K delta and gamma inhibitor to the site of action, such as orally, intranasally, topically (e.g., transdermally), intraduodenally, parenterally (including intravenously, intraarterially, intramuscularally, intravascularally, intraperitoneally or by injection or infusion), intradermally, by intramammary, intrathecally, intraocularly, retrobulbarly, intrapulmonary (e.g., aerosolized drugs) or subcutaneously (including depot administration for long term release e.g., embedded-under the-splenic capsule, brain, or in the cornea), sublingually, anally, rectally, vaginally, or by surgical implantation (e.g., embedded under the splenic capsule, brain, or in the cornea).

The pharmaceutical compositions described herein can be administered in solid, semi-solid, liquid or gaseous form, or may be in dried powder, such as lyophilized form. The pharmaceutical composition can be packaged in forms convenient for delivery, including, for example, solid dosage forms such as capsules, sachets, cachets, gelatins, papers, tablets, suppositories, pellets, pills, troches, and lozenges. The type of packaging will generally depend on the desired route of administration. Implantable sustained release formulations are also contemplated, as are transdermal formulations.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages.

Oral solid dosage forms are described in, e.g., Remington's Pharmaceutical Sciences, 20th Ed., Lippincott Williams & Wilkins, 2000, Chapter 89, “Solid dosage forms include tablets, capsules, pills, troches or lozenges, and cachets or pellets”. Also, liposomal or proteinoid encapsulation may be used to formulate the compositions (as, for example, proteinoid microspheres reported in U.S. Pat. No. 4,925,673). Liposomal encapsulation may include liposomes that are derivatized with various polymers (e.g., U.S. Pat. No. 5,013,556). The pharmaceutical compositions described herein may include a dual selective PI3K delta and gamma inhibitor and inert ingredients which protect against degradation in the stomach and which permit release of the biologically active material in the intestine.

The amount of the dual selective PI3K delta and gamma inhibitor, such as Compound (A) or a pharmaceutically acceptable salt thereof, to be administered is dependent on the mammal being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.05 to about 2.5 g/day An effective amount of a compound of the invention may be administered in either single or multiple doses (e.g., two or three times a day).

The term “co-administration,” “administered in combination with,” and their grammatical equivalents, as used herein, encompasses administration of two or more agents to a subject so that both agents and/or their metabolites are present in the animal at the same time. Co-administration includes simultaneous administration in separate compositions, administration at different times in separate compositions, or administration in a composition in which both agents are present.

More preferably, the dual selective PI3K delta and gamma inhibitor is Compound (A) or a pharmaceutically acceptable salt thereof.

A further embodiment of the present invention relates to a method of treating peripheral T-cell lymphoma (PTCL) and cutaneous T-cell lymphoma (CTCL) comprising administering a therapeutically effective amount of a pharmaceutical composition as described herein to a subject (preferably, a human subject) in need thereof.

A further embodiment of the present invention relates to the use of a pharmaceutical composition as described herein in the preparation of a medicament for treating PTCL or CTCL.

The following general methodology described herein provides the manner and process of using the dual selective PI3K delta and gamma inhibitor and are illustrative rather than limiting. Further modification of provided methodology and additionally new methods may also be devised in order to achieve and serve the purpose of the invention. Accordingly, it should be understood that there may be other embodiments which fall within the spirit and scope of the invention as defined by the specification hereto

### Routes of Administration

In any of the methods and uses described herein, the dual selective PI3K delta and gamma inhibitor and pharmaceutical composition may be administered by various routes. For example, the dual selective PI3K delta and gamma inhibitor and pharmaceutical composition may be formulated for injection, or for oral, nasal, transdermal or other forms of administration, including, e.g., by intravenous, intradermal, intramuscular, intramammary, intraperitoneal, intrathecal, intraocular, retrobulbar, intrapulmonary (e.g., aerosolized drugs) or subcutaneous injection (including depot administration for long term release e.g., embedded-under the-splenic capsule, brain, or in the cornea), by sublingual, anal, or vaginal administration, or by surgical implantation, e.g., embedded under the splenic capsule, brain, or in the cornea. The treatment may consist of a single dose or a plurality of doses over a period of time. In general, the uses and methods described herein may involve administering an effective amount of a dual selective PI3K delta and gamma inhibitor (such as Compound (A) or a pharmaceutically acceptable salt thereof) together with one or more pharmaceutically acceptable diluents, preservatives, solubilizers, emulsifiers, adjuvants and/or carriers, as described above.

The present invention is now further illustrated by means of biological examples.

### Example 1

### Anti-Proliferative Effect of Compound (A) in T-Cell Lymphoma Cell Lines (MTT Assay)

Compound (A) was tested across a panel of T-cell lymphoma cell lines (Jurkat, MOLT-4, CCRF-CEM, HuT-78, HuT-102, Sez4 and HH). Cells were plated in 96-well plates and incubated with desired concentrations of Compound A for 48-72 h. At the end of the incubation period, MTT ((3-(4,5-dimethylthiazol-2-yl)-2,5-diphenyltetrazolium bromide)) was added. The plate was placed on a shaker for 5 min to mix the formazan and the optical density at 560 nM was measured on a spectrophotometer. Data were plotted using Graphpad prism for calculation of the IC₅₀ concentrations.

AKT, a serine threonine kinase mediates the downstream effects of PI3K activity and modulates several cell processes including survival and growth. Reduction of pAKT by Compound (A) in representative cell lines was determined by Western blotting using a phospho-AKT (Ser⁴⁷³) antibody. Band intensity was measured and quantified using ImageJ software and normalized to actin.

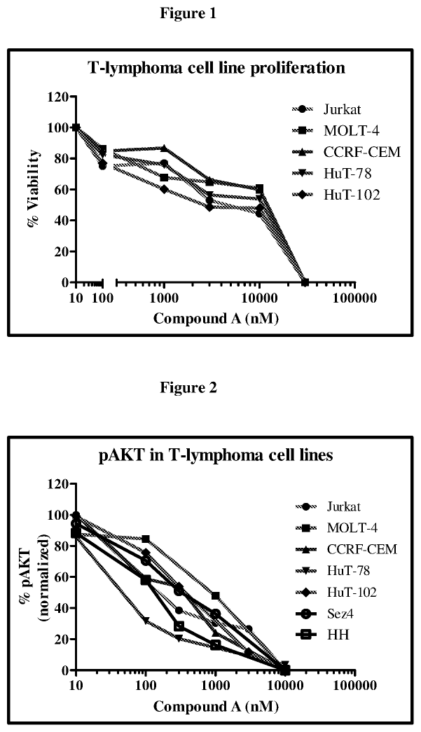

Results: Compound (A) demonstrated inhibition of growth (FIG. 1) and Phospho-AKT (FIG. 2) in the T-lymphoma cell lines. Compound (A) caused a dose-dependent reduction in proliferation and endogenous pAKT expression in all T-cell lymphoma cell lines.

### Example 2

### Induction of Caspase 3 by Compound (A)

Cells (Jurkat, MOLT-4, CCRF-CEM, HuT-78 and HuT-102) were incubated with desired concentrations of Compound (A) for 48 h. An equal number of cells per well (0.3×10⁶ cells) were used. Increase in apoptosis manifested by an elevation in caspase-3 levels was determined using a Caspase-3 kit from Millipore. Induction of Caspase 3 by Compound A was measured fluorimetrically.

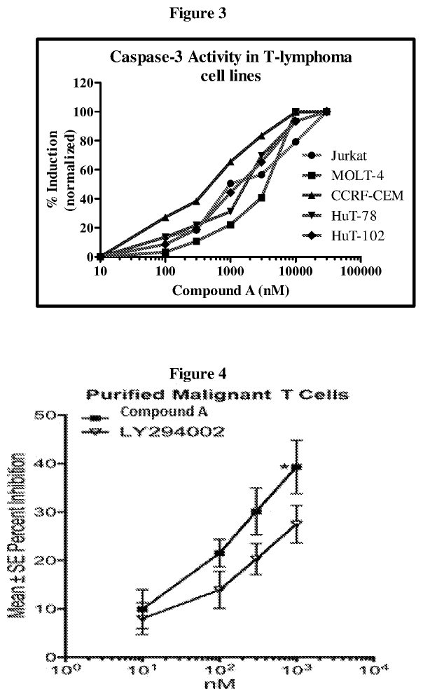

Results: A dose-dependent increase in caspase-3 was observed with Compound (A) (FIG. 3).

### Example 3

### Effect of Compound (A) on Patient Derived Primary Cells

The effect of Compound (A) on pAKT in patient-derived primary cells was also studied. Malignant T cells from Cutaneous T-cell Lymphoma (CTCL) patient donors (n=6) were purified using fluorescence-activated cell sorting (FACS) and cultured overnight in RPMI/1% BSA. Cells were incubated with desired concentrations of Compound (A) for 1.5 h followed by activation with a cytokine mixture (20 ng/ml IL2+5 ng/ml IL7+10 ng/ml IL15+10% FBS) for 30 min. pAKT was estimated using Phosphoflow and normalized to total AKT. Data were analyzed using Prism 5.0 software analysis. For apoptosis assays, FACS purified cells from CTCL donors (n=4) were cultured in RPMI/10% FBS+20 ng/ml IL2+5 ng/ml IL7+10 ng/ml IL15 with and without Compound (A), LY294002, or camptothecin for 48 h. Apoptosis was assayed by Annexin V/PI staining.

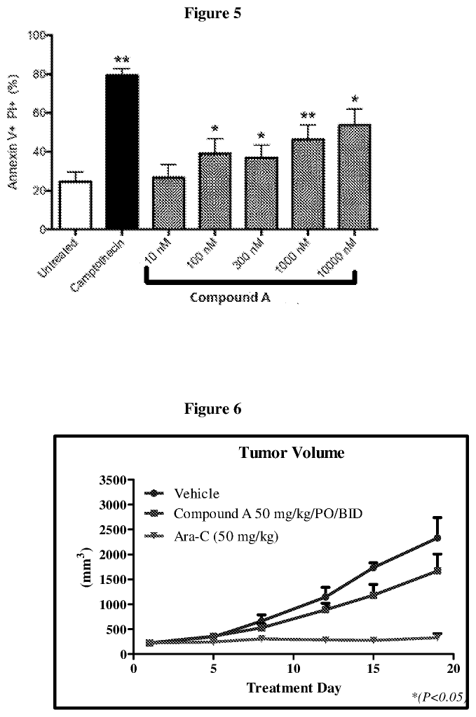

Results: Compound (A) demonstrated dose-dependent inhibition of pAKT (FIG. 4) and dose-dependent increases in apoptosis (FIG. 5) in purified malignant T-cells.

### Example 4

### The Anti-Tumor Effects of Compound (A) in T-Cell Lymphoma Xenograft

The anti-tumor effect of Compound (A) was determined in a MOLT-4 (representing human T lymphoblast cell line) subcutaneous mouse xenograft model. Briefly, 10⁶ cells were injected into the flank region. Mice were randomized according to body weight into two groups of five. A week after tumor cell injection, mice either received the vehicle, oral administration of Compound (A) at 50 mg/kg/BID Compound (A), or administration of cytarabine (Ara-C), over an 18-day study period. At the end of the study period, animals were sacrificed and the tumors harvested.

Data revealed that the mice tolerated the daily dose of 50 mg/kg Compound (A) without body weight loss or noticeable adverse effects. At the dose tested, Compound (A) significantly delayed tumor growth compared to vehicle treated control group.

Results: Compound (A) demonstrated significant delay in tumor growth in the MOLT-4 Human Leukemia Xenograft Model (FIG. 6).

### Example 5

### Effect of Compound (A) on PTCL & CTCL Patients

Trial Design

This is a Phase I/Ib, 3+3 design study in patients with relapsed or refractory T-cell lymphoma

Compound (A) (tenalisib) was given orally twice a day in 28-day cycles and dose-limiting toxicities (DLTs) were assessed during the first cycle.

Intra-patient dose escalation was allowed following safety of higher doses.
Primary Objectives: The Safety, Pharmacokinetics (PK), Maximum Tolerated Dose (MTD)
Secondary Objectives: Pharmacodynamics, Overall response rate (ORR), Duration of response (DOR)

Key Eligibility Criteria

Histologically confirmed T-cell Non-Hodgkin's lymphoma.

Relapsed after, or refractory to ≥1 prior treatments, and not eligible for transplantation and or approved therapy; ECOG performance status ≤2; patient with measurable or evaluable disease; Adequate organ system function: ANC ≥750/μL; platelets ≥50 K/μL.

Prior therapy that inhibits PI3K/BTK/mTOR were part of exclusion criteria.

The patient demographics are provided below.

### Patient Demographics

| DEMOGRAPHICS | PTCL(n = 28) | | CTCL (n = 30) | | All (n = 58) | |
|---|---|---|---|---|---|---|
| Age (years), Median (Range) | 63 | (40-89) | 68 | (39-84) | 66.5 | (39-89) |
| Gender | | | | | | |
| Male, n (%) | 17 | (61) | 13 | (43) | 30 | (52) |
| Female, n (%) | 11 | (49) | 17 | (57) | 28 | (48) |
| Prior therapies, Median (Range) | 3 | (1-7) | 5.5 | (2-15) | 4 | (1-15) |
| Patients with ≥3 therapies, n (%) | 17 | (61) | 26 | (87) | 43 | (74) |
| Patients with ≥5 therapies, n (%) | 6 | (21) | 19 | (63) | 25 | (43) |
| Stage, 3 or 4, n, (%) | 26 | (93) | 15 | (50) | 41 | (71) |
| ECOG, 0/1/2 | 20/8/0 | | 26/4/0 | | 46/12/0 | |
| Disease status | | | | | | |
| Relapse, n (%) | 18 | (64) | 13 | (43) | 31 | (53) |
| Refractory, n (%) | 10 | (36) | 17 | (57) | 27 | (47) |

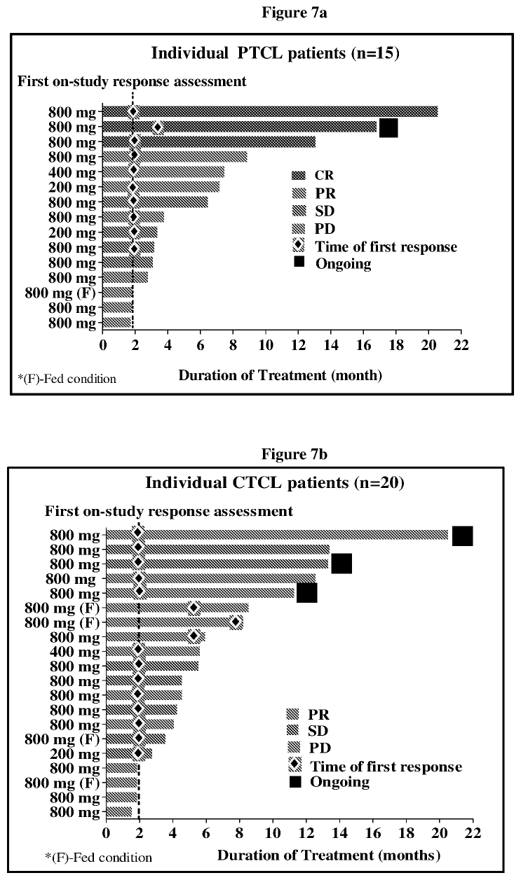

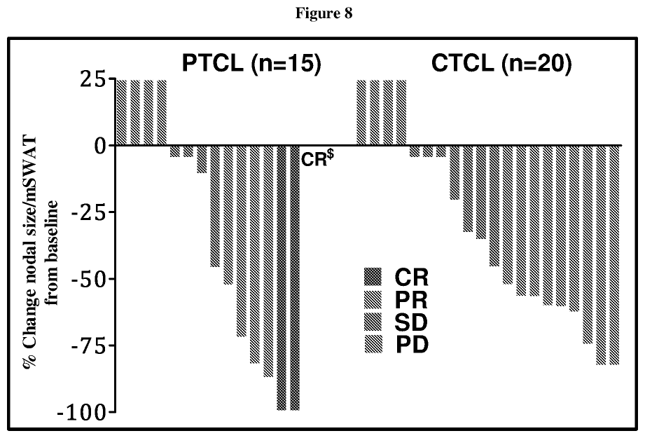

Results: Anti-tumor activity of Compound (A) is shown in FIGS. 7a, 7b and 8. The results are provided in Table 1 below where the duration of treatment in efficacy evaluable patients PTCL (n=15) and CTCL (n=20) is shown.

TABLE 1

| | Patients | | | | | | | | | | DCR |
|---|---|---|---|---|---|---|---|---|---|---|---|
| | Treated/ | Best Observed Response | | | | | | | | | (CR + |
| Pop- | Evaluable | n (%) | | | | | | | | | PR + |
| ulation | (n) | ORR | | CR | | PR | | SD | | PD | SD) |
| All | 58/35 | 16 | (46) | 3 | (9) | 13 | (37) | 11 | (31) | 8 (23) | 26 (74) |
| PTCL | 28/15 | 7 | (47) | 3 | (20) | 4 | (27) | 4 | (27) | 4 (27) | 10 (74) |

| | | | | | | | | | | |
|---|---|---|---|---|---|---|---|---|---|---|
| CTCL | 30/20 | 9 | (45) | — | 9 | (45) | 7 | (35) | 4 (20) | 16 (80) |

ORR = Objective response rate;
CR = complete response;
PR = partial response;
SD = stable disease;
PD = progressive disease;
DCR = disease control rate

23 patients (13 PTCL; 10 CTCL) were not considered for efficacy analysis due to rapid disease progression as per the protocol.

Median duration of treatment: PTCL [1.9 months (0.4, 20.67)], CTCL [3.45 months (0.7, 20.56)]

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as described above. It is intended that the above description define the scope of the invention and that methods and structures within the scope of these description and their equivalents be covered thereby.

All publications and patent and/or patent applications cited in this application are herein incorporated by reference to the same extent as if each individual publication or patent application was specifically and individually indicated to be incorporated herein by reference.

1. A method of treating peripheral T-cell lymphoma (PTCL) or cutaneous T-cell lymphoma (CTCL) comprising administering to a subject (S)-2-(1-(9H-purin-6-ylamino)propyl)-3-(3-fluorophenyl)-4H-chromen-4-one or a pharmaceutically acceptable salt thereof.

2. The method of claim 1, wherein the subject suffers from cutaneous T-cell lymphoma (CTCL).

3. The method of claim 45, wherein (S)-2-(1-(9H-purin-6-ylamino)propyl)-3-(3-fluorophenyl)-4H-chromen-4-one or a pharmaceutically acceptable salt thereof is administered as a front-line therapy for the peripheral T-cell lymphoma (PTCL).

4. The method of claim 45, wherein the subject suffers from relapsed-refractory peripheral T-cell lymphoma (PTCL).

5. The method of claim 2, wherein the (S)-2-(1-(9H-purin-6-ylamino)propyl)-3-(3-fluorophenyl)-4H-chromen-4-one or a pharmaceutically acceptable salt thereof is administered as a front-line therapy for the cutaneous T-cell lymphoma (CTCL).

6. The method of claim 2, wherein the subject suffers from relapsed-refractory cutaneous T-cell lymphoma (CTCL).

7. The method of claim 1, wherein the subject is human.

8. The method of claim 1, wherein the (S)-2-(1-(9H-purin-6-ylamino)propyl)-3-(3-fluorophenyl)-4H-chromen-4-one or a pharmaceutically acceptable salt thereof is administered to the subject by the oral, intravenous, intramuscular, or intraperitoneal route.

9. The method of claim 8, wherein the (S)-2-(1-(9H-purin-6-ylamino)propyl)-3-(3-fluorophenyl)-4H-chromen-4-one or a pharmaceutically acceptable salt thereof is administered by the oral route.

10. The method of claim 1, wherein the (S)-2-(1-(9H-purin-6-ylamino)propyl)-3-(3-fluorophenyl)-4H-chromen-4-one or a pharmaceutically acceptable salt thereof is administered at a dose of
i) about 25 to about 2000 mg,
ii) about 25 to about 1600 mg,
iii) about 25 to about 1200 mg,
iv) about 25 to about 800 mg,
v) about 25 to about 600 mg, or
vi) about 25 to about 400 mg.

11. The method of claim 10, wherein the dose is
i) about 50 to about 2000 mg,
ii) about 50 to about 1600 mg,
iii) about 50 to about 1200 mg,
iv) about 50 to about 800 mg,
v) about 50 to about 600 mg, or
vi) about 50 to about 400 mg.

12. The method of claim 10, wherein the dose is
i) about 200 to about 2000 mg,
ii) about 200 to about 1600 mg,
iii) about 200 to about 1200 mg,
iv) about 200 to about 800 mg,
v) about 200 to about 600 mg, or
vi) about 200 to about 400 mg.

13. The method of claim 10, wherein the dose is
i) about 400 to about 2000 mg,
ii) about 400 to about 1600 mg,
iii) about 400 to about 1200 mg,
iv) about 400 to about 800 mg, or
v) about 400 to about 600 mg.

14. The method of claim 1, wherein the (S)-2-(1-(9H-purin-6-ylamino)propyl)-3-(3-fluorophenyl)-4H-chromen-4-one or a pharmaceutically acceptable salt thereof is administered as a single or in divided doses.

15. The method of claim 1, further comprising administering one or more anti-cancer treatments, one or more cytostatic, cytotoxic or anticancer agents, targeted therapy, or any combination of any of the foregoing.

16. The method of claim 15, wherein the (S)-2-(1-(9H-purin-6-ylamino)propyl)-3-(3-fluorophenyl)-4H-chromen-4-one or a pharmaceutically acceptable salt thereof is administered together or sequentially with the one or more anti-cancer treatments, one or more cytostatic, cytotoxic or anticancer agents or targeted therapy.

17. The method of claim 15, wherein the anticancer agents are selected from DNA interactive agents, alkylating agents, topoisomerase II inhibitors, topoisomerase I inhibitors, tubulin interacting agents, hormonal agents, thymidilate synthase inhibitors, anti-metabolites, tyrosine kinase inhibitors, angiogenesis inhibitors, EGF inhibitors, VEGF inhibitors, CDK inhibitors, SRC inhibitors, c-Kit inhibitors, Her1/2 inhibitors, checkpoint kinase inhibitors, monoclonal antibodies directed against growth factor receptors selected from EGF and Her2, CD20 monoclonal antibodies, B-cell targeting monoclonal antibodies, fusion proteins, protein kinase modulators, CHOP (cyclophosphamide, doxorubicin, vincristine, prednisone), R-CHOP (rituximab-CHOP), hyperCV AD (hyperfractionated cyclophosphamide, vincristine, doxorubicin, dexamethasone, methotrexate, cytarabine), R-hyperCV AD (rituximab-hyperCV AD), FCM (fludarabine, cyclophosphamide, mitoxantrone), R-FCM (rituximab, fludarabine, cyclophosphamide, mitoxantrone), bortezomib and rituximab; temsirolimus and rituximab, temsirolimus and bortezomib, Iodine-131 tositumomab and CHOP, CVP (cyclophosphamide, vincristine, prednisone), R-CVP (rituximab-CVP), ICE (iphosphamide, carboplatin, etoposide), R-ICE (rituximab-ICE), FCR (fludarabine, cyclophosphamide, rituximab), FR (fludarabine, rituximab), and D.T. PACE (dexamethasone, thalidomide, cisplatin, adriamycin, cyclophosphamide, etoposide), steroidal anti-inflammatory drugs, non-steroidal anti-inflammatory drugs (NSAIDs), immune selective anti-inflammatory derivatives (ImSAIDs), anti-emetic, analgesic, anti-inflammatory, anti-cachexia agents, or any combination of any of the foregoing.

18. The method of claim 15, wherein the anticancer treatment is selected from chemotherapy, radiation therapy, biological therapy, bone marrow transplantation, stem cell transplant, or any combination of any of the foregoing.

19-44. (canceled)

45. The method of claim 1, wherein the subject suffers from peripheral T-cell lymphoma (PTCL).
